(19) United States
(12) Patent Application Publication
(10) Pub. No.: US 2016/0361490 A1
(43) Pub. Date: Dec. 15, 2016

(54) SAFETY NEEDLE ASSEMBLIES AND RELATED METHODS

(71) Applicant: B. Braun Melsungen AG, Melsungen (DE)

(72) Inventors: Chee Mun Phang, Penang (MY); Siew Ping Lim, Penang (MY); Riduan bin Tajudin, Penang (MY); Soo Yong Tan, Penang (MY)

(73) Assignee: B. Braun Melsungen AG, Melsungen (DE)

(21) Appl. No.: 14/737,960

(22) Filed: Jun. 12, 2015

(51) Int. Cl. A61M 5/162; A61M 25/00; A61M 5/36; A61M 25/06

(52) U.S. Cl. CPC A61M 5/1626; A61M 25/0606; A61M 25/0075; A61M 25/0097; A61M 25/0014; A61M 25/001; A61M 25/0015; A61M 5/36; A61M 2205/0216

(57) ABSTRACT

Safety needle assemblies that include a needle having a sharp distal tip and a needle tip cover mounted on the needle are disclosed. The needle tip cover includes a first portion having a channel receiving the needle and a second portion attached to the first portion. The second portion has a proximal wall, an opening in the proximal wall receiving the needle, a sidewall extending distally from the proximal wall, first and second spring arms extending transversely from opposite edges of the sidewall, and a third spring arm extending distally from the sidewall. The first spring arm and the second spring arm extend at least partially around the first portion. The third spring arm includes a flange extending radially inward and configured to bear against the needle to deflect the third spring arm radially outward against a spring restoring force in a ready to use position.

### FIELD OF ART

The present invention is generally directed to needle safety assemblies and related methods and more particularly to needle safety assemblies and related methods utilizing needle guards.

### BACKGROUND

Insertion procedure for an intravenous (IV) catheter assembly includes the following four basic steps: (1) the healthcare worker inserts the needle and catheter together into the patient's vein; (2) after insertion into the vein with the needle point, the catheter is advanced into the vein of the patient by the healthcare worker pushing the catheter hub with his or her finger; (3) the healthcare worker withdraws the needle by grasping the needle hub end (opposite the point end) while at the same time applying pressure to the patient's skin at the insertion site with his or her free hand to stop the flow of blood through the catheter; and (4) the healthcare worker then tapes the exposed end of the catheter (the catheter hub) to the patient's skin and connects it to the source of the fluid to be administered into the patient's vein.

One potential problem with traditional procedure is that immediately after the withdrawal of the needle from the patient's vein, the healthcare worker, who is at this time involved in at least two urgent procedures, must place the exposed needle tip at a nearby location and address the tasks required to accomplish the needle withdrawal. It is at this juncture that the exposed needle tip creates a danger of an accidental needle stick, which, under the circumstances, can leave the healthcare worker vulnerable to the transmission of various dangerous blood-borne pathogens, including AIDS and hepatitis.

Other needle types similarly expose healthcare workers to risks of accidental needle sticks. For example, a doctor administering an injection using a straight needle, a Huber needle, an epidural needle, etc., may place the used needle on a tray for subsequent disposal by a nurse. For the period between placing the used needle on a tray or a work station to the time it is discarded, the used needle is a potential source for disease transmissions for those that work near or around the needle.

### SUMMARY

The various embodiments of a needle assembly have several features, no single one of which is solely responsible for their desirable attributes. Without limiting the scope of the present embodiments as set forth in the claims that follow, their more prominent features now will be discussed briefly.

A catheter assembly provided in accordance with aspects of the present disclosure can comprise a catheter tube extending distally from a distal end of a catheter hub, a needle hub having an interior cavity, a needle guard having a lengthwise axis and comprising a sleeve having a proximal end attached to the needle hub and a tip cover attached at a distal end of the sleeve, the sleeve surrounding at least a portion of the needle and the tip cover comprising a nose section and a perimeter defining a side opening located off-axis from the lengthwise axis of the needle guard, a needle with a needle tip projecting through the sleeve, the side opening of the tip cover, the catheter hub, and the catheter tube in a ready to use position with the needle tip extending distally of a distal opening of the catheter tube.

A valve can be positioned in an interior cavity of the catheter hub.

The catheter hub can be a ported catheter with an injection port.

A port cap assembly can cap the injection port of the ported catheter.

The valve can have a port valve sealing the injection port from an interior cavity of the catheter hub, and an inlet valve sealing fluid flow from a proximal opening of the catheter hub.

The sleeve can be elastic and compressed in the ready to use position.

The sleeve can be compressed and bent near a distal end of the sleeve.

The sleeve can be made of a compressible syntactic rubber, neoprene, or isoprene.

The sleeve can be attached to the needle hub via a ring fixed to the proximal end of the sleeve and inside a distal shroud of the needle hub.

The tip cover can slide relative to the needle from the ready to use position to a secured position where the needle tip enters into the tip cover through the side opening.

The side opening can have a width larger than a diameter of the needle.

A perimeter of the side opening can be smooth to minimize friction between the needle and the tip cover.

The tip cover can abut against the catheter hub in the ready to use position.

The tip cover can abut against the catheter hub and the sleeve can be attached to the needle hub in the ready position and during retraction of the needle away from the catheter hub.

The tip cover can further comprise a flap extending in a proximal direction inside the tip cover from a distal end of the side opening, said flap being configured for preventing the needle tip from emerging out of the side opening from the secured position.

The tip cover can be made of metal. Alternatively, the tip cover can be made from POM (polyoxymethylene) or ABS (acrylonitrile butadiene styrene).

The tip cover can be tethered to the needle hub.

The tether can be a folded mechanism attached to the tip cover and the needle hub.

The folded mechanism can be attached to a nose plate, which is attached to the tip cover.

The folded mechanism can comprise a plurality of legs folded in the interior cavity of the needle hub in the ready to use position.

The tip cover and the folded mechanism can be integrally formed.

An air vent housing can be attached to a proximal end of the needle hub, a closing cap attached to a proximal end of the air vent housing, and a filter sandwiched between the air vent housing and the closing cap.

A further aspect of the present disclosure can include a method of making a catheter assembly which can comprise providing a catheter unit comprising a catheter hub with a hub body, a catheter tube extending distally from a distal end of the catheter hub, and a valve positioned in an interior cavity of the catheter hub, providing a needle hub unit comprising a needle hub having an interior cavity, a needle with a needle tip extending distally from the needle hub, and a needle guard having a lengthwise axis coupled to the needle hub, said needle guard comprising an elastic sleeve having a proximal end coupled to the needle hub, and a tip cover attached to a distal end of the sleeve, said tip cover having a nose section and a side opening located off-axis from the lengthwise axis of the needle guard, extending the needle through the elastic sleeve and out the side opening on the tip cover, through the valve, and through the catheter tube with the needle tip extending distally of a distal opening of the catheter tube in a ready to use position, compressing the elastic sleeve against the catheter hub and the needle hub in the ready to use position, and tilting the nose section of the tip cover from the lengthwise axis of the needle guard.

A side injection port can extend from the hub body.

A port cap assembly can cap an injection port.

The method can further comprise sliding the tip cover relative to the needle from the ready to use position to the secured position.

The sleeve can be attached to the needle hub via a ring at the proximal end of the sleeve and the distal nose section in the interior cavity of the needle hub.

The tip cover can be tethered to the needle hub.

The tip cover can be tethered with a folded mechanism that is attached to a flange extending from the tip cover and to a heat weld nipple extending from the needle hub.

The folded mechanism can be attached to a nose plate which is coupled to the tip cover.

The nose plate can have a perimeter defining an opening having the tip cover projecting therethrough.

The tip cover and the folded mechanism can be integrally formed.

The tip cover can further comprise a flap extending proximally inside the tip cover from a distal end of the side opening, said flap being configured for preventing the needle tip from coming out of the side opening.

The side opening can have a width larger than a diameter of the needle.

A perimeter of the side opening and a perimeter of the opening is smooth to minimize friction between the needle and the tip cover.

The tip cover can abut against the catheter hub in the ready to use position.

The tip cover can further comprise a flap extending proximally inside the tip cover from a distal end of the side opening, said flap being configured for preventing the needle tip from coming out of the side opening.

The method can further comprise providing an air vent housing attached to a proximal end of the needle hub, a closing cap attached to a proximal end of the air vent housing, and a filter sandwiched between the air vent housing and the closing cap.

Aspects of the present disclosure include a ported catheter IV assembly that includes a ported catheter comprising a catheter hub, a catheter tube extending distally from a distal end of the catheter hub, and a valve positioned in an interior cavity of the catheter hub, and a needle hub unit comprising a needle hub having an interior cavity, a needle extending distally from the interior cavity of the needle hub, said needle extending through the valve and the catheter tube with a needle tip extending distally of a distal opening of the catheter tube in a ready to use position, and a needle guard coupled to the needle hub.

The needle guard can comprise an elastic sleeve having a proximal end coupled to the needle hub, and a tip cover attached at a distal end of the sleeve. The sleeve is sleeved over at least a portion of the needle. The tip cover has an opening at a proximal end and a slotted hole.

The needle extends through the sleeve, the opening of the tip cover, and the slotted hole of the tip cover in the ready to use position, and the needle tip is restrained inside the tip cover in a secured position.

The ported catheter can further comprise a port cap assembly capping an injection port of the ported catheter.

The sleeve can be elastic and compressible against a restoring force of the sleeve in the ready to use position.

The sleeve can be attached to the needle hub via a ring fixed to the proximal end of the sleeve and the interior cavity of the needle hub.

The tip cover can slide along the needle from the ready to use position to the secured position when the needle is retracted proximally from the ported catheter.

The tip cover can be tethered to the needle hub via a folded mechanism.

The folded mechanism can be tethered to a flange extending from the tip cover and a heat weld nipple extending from the needle hub.

The folded mechanism can comprise a nose plate and a pair of legs extending from the nose plate to the needle hub, said nose plate defining a hole receiving the tip cover.

The tip cover and the folded mechanism can be integrally formed.

The tip cover further can comprise a flap extending proximally inside the tip cover from a distal end of the slotted hole, said flap being configured for preventing the needle tip from coming out of the slotted hole.

The ported IV catheter assembly can further comprise an air vent housing attached to a proximal end of the needle hub, a closing cap attached: to a proximal end of the air vent housing, and a filter sandwiched between the air vent housing and the closing cap.

Another aspect of the present disclosure includes a method of making a ported IV catheter assembly, the method comprising providing a ported catheter comprising a catheter hub, a catheter tube extending distally from a distal end of the catheter hub, and a valve positioned in an interior cavity of the catheter hub.

The method can further comprise providing a needle hub unit comprising a needle hub having an interior cavity, a needle extending distally from the interior cavity of the needle hub, and a needle guard coupled to the needle hub, said needle guard comprising an elastic sleeve having a proximal end coupled to the needle hub, and a tip cover attached at a distal end of the sleeve, said tip cover having an opening at a proximal end and a slotted hole.

The method can further comprise extending a needle tip of the needle through the elastic sleeve, the opening of the tip cover and out the slotted hole, the valve, and the catheter tube with the needle tip extending distally of a distal opening of the catheter tube in a ready to use position.

The method can further comprise compressing the elastic sleeve against the catheter hub in the ready to use position.

The method can further comprise confining the needle tip inside the tip cover in a secured position.

The ported catheter can further comprise a port cap assembly capping an injection port of the ported catheter.

The method can further comprise sliding the tip cover along the needle from the ready to use position to the secured position.

The sleeve can be attached to the needle hub via a ring fixed to the proximal end of the sleeve and the interior cavity of the needle hub.

The tip cover can be tethered to the needle hub via a folded mechanism.

The folded mechanism can be tethered to a flange extending from the tip cover and a heat weld nipple extending from the needle hub.

The folded mechanism can comprise a nose plate and a pair of legs extending from the nose plate to the needle hub, said nose plate defining a hole receiving the tip cover.

The tip cover and the folded mechanism can be integrally formed.

The tip cover can further comprise a flap extending proximally inside the tip cover from a distal end of the slotted hole, said flap being configured for preventing the needle tip from coming out of the slotted hole.

### BRIEF DESCRIPTION OF THE DRAWINGS

The various embodiments of the present safety needle assemblies and needle tip covers now will be discussed in detail with an emphasis on highlighting the advantageous features. These embodiments depict the novel and non-obvious safety needle assemblies and needle tip covers shown in the accompanying drawings, which are for illustrative purposes only. These drawings include the following figures, in which like numerals indicate like parts:

FIG. 1 is an assembled isometric view of an embodiment of a safety IV needle assembly including a cannula assembly and a catheter assembly;

FIG. 2 is an exploded perspective view of the cannula assembly and the catheter assembly of FIG. 1;

FIG. 3 is a partial perspective view of a needle guard with a tip cover and a sleeve;

FIG. 4 is an assembled side cross-sectional view of the safety IV needle assembly of FIG. 1;

FIG. 5 is a partial cutaway perspective view of the safety IV needle assembly of FIG. 1;

FIG. 6 is a close up view of the safety IV needle assembly of FIG. 5 in perspective and separated from the catheter hub;

FIG. 7 is a perspective view of the needle hub unit removed from the catheter hub unit, such as following successful venipuncture, to show the needle guard shielding the needle tip;

FIG. 8 is a cross-sectional side view of the needle hub unit in a shielded position;

FIG. 9 is a close up view of circled region 9 of the needle hub unit if FIG. 8;

FIG. 10 is a close up view of a tip cover of the present disclosure;

FIG. 11 is a perspective view of another embodiment of a tip cover of the cannula assembly;

FIG. 12 is a close up view of a catheter assembly with a needle guard in accordance with an alternative embodiment; and

FIG. 13 is a close up view of the needle guard of FIG. 12 in a shielded position.

### DETAILED DESCRIPTION

The following detailed description describes the present devices, apparatuses, systems, and methods with reference to the drawings. In the drawings, reference numbers label elements of the present embodiments. These reference numbers are reproduced below in connection with the discussion of the corresponding drawing features.

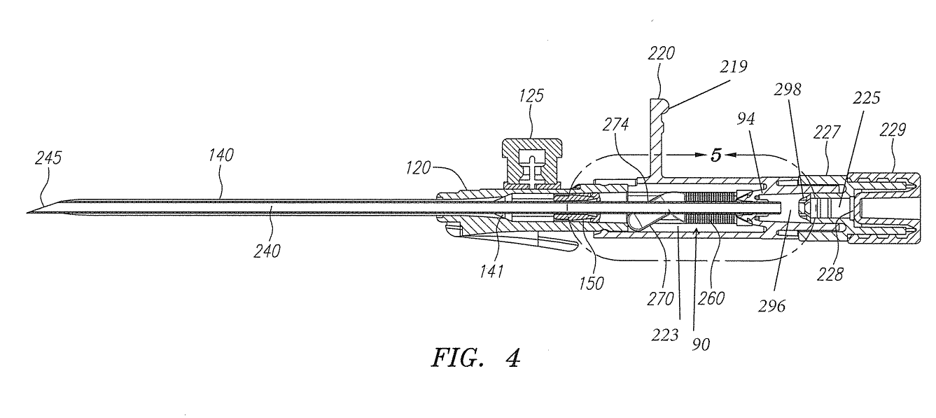

With reference now to Figures (“FIGS.”) 1 and 2, an embodiment of an IV catheter assembly 10 is shown comprising a ported catheter or ported catheter unit 100 and a needle hub unit 200 detachably assembled to a proximal end of the ported catheter 100 in a ready to use position. In an example, the ported catheter unit 100 comprises a catheter hub 120 having a catheter body 121 and an injection port 124 extending from the catheter body 121, a port cap assembly 125 capping the injection port 124, a valve 150 (FIGS. 3 and 4) can be disposed in the interior cavity 127 of the catheter body 121 to control or restrict the flow of fluids through the injection port 124 and through the proximal opening at a proximal end 126 of the catheter hub 120, and a catheter tube 140 extending distally from a distal end 123 of the catheter hub 120.

Conventionally speaking, the distal end 123 of the catheter hub 120 is the end away from the practitioner, such as toward the tip of the catheter tube 140, and the proximal end 126 is the end opposite the distal end, typically closer to the practitioner. In alternative catheter embodiments, the injection port 124 may be omitted. A distal end of the catheter tube 140 is configured for insertion into the patient and a proximal end of the catheter tube 140 can be attached to the catheter body 121 by a bushing 141. The bushing 141 is attached at to the catheter body 121 at the distal end 123 thereof inside the interior cavity 127 to secure the catheter tube 140 to the catheter body 121. The catheter hub 120 is configured for use with an intravenous tubing at a proximal end 126 thereof to facilitate delivery of fluids into the patient. For example, when the catheter 140 is inserted into the patient, solution such as saline solution and/or medication can flow from the proximal end 126 of the catheter hub 120 to the catheter tube 140 without leakage. Fluid samples can also be drawn from the patient through the proximal end 126 of the catheter hub 120. In an example, the proximal opening of the catheter hub 120 is shaped as a female Luer, with or without external threads. As shown, proximal end of the catheter hub 120 has external threads for receiving a male Luer tip, such as a syringe tip or an IV adaptor, with or without a threaded collar for engaging the external threads.

As shown, the catheter hub 120 has a hub lengthwise axis and the injection, port 124 has a port lengthwise axis that is generally perpendicular to and located off-axis to the hub lengthwise axis. In other words, the two angled axes do not intersect. In another example, the axis of the injection port 124 intersects the axis of the catheter hub 120, such as being in-line, and preferably at a right angle therefrom, as further discussed below. An optional pair of wings 129 extends from the catheter body 121 for use to secure and stabilize the ported catheter unit 100 to the patient following catheterization.

The interior cavity 127 of the catheter body 121 is selectively in fluid communication with the injection port 124 depending on the status or configuration of the valve 150. The female Luer connector of the catheter hub is thus configured to matingly receive a male Luer connector, such as an IV line, a Luer access connector, a syringe tip, a vent plug, other known connectors, or future-developed IV devices. Each of these components can be sized and configured in conformity with at least some of the International Standards Organization (ISO) standards for female and male Luer connections under current or future standards. For discussion purposes, any one of these components or the class of these components may be referred to as a male medical implement.

In an example, the catheter body 121 has a latching means for engaging with and removably securing the needle hub unit 200 to the catheter hub 120. In an embodiment, the latching means comprises one or more recesses 122 formed on an exterior of the catheter body 121, at or near the proximal end 126 of the catheter hub 120 to engage corresponding bumps or projections on the needle hub. The recesses and the bumps can be located in reversed order. In other embodiments, other engagement means, such as male or female detents, for removably attaching the needle hub unit 200 to the proximal end of the ported catheter 100, are contemplated.

With reference to FIGS. 3 and 4, the valve 150 is configured to seal the internal opening of the injection port 124 from the interior cavity 127 of the catheter body 121 and also seals the proximal opening 126 of the catheter body 121 to prevent flow through the valve 150 and through the catheter hub from the proximal opening of the catheter hub. In an example, the valve 150 is made from an elastomeric or a thermoplastic elastomer (TPE) material and includes two valve components or valve sections, namely a port valve 151 and an inlet valve 149 comprising a septum. The septum of the inlet valve 149 seals the proximal opening at the proximal end 126 of the catheter body 121 and opens into or leads into the interior cavity 127 of the catheter hub 120. The elongated body of the port valve 151 is configured to occlude the internal opening of the injection port 124 from the interior cavity 127 to block fluid flow coming from or passing through the injection port 124. Thus, the valve 150, because it has a port valve 151 component and an inlet valve 149 component, may be referred to as a combination valve, a dual function valve, or a combined valve. In one example, the port valve 151 and the inlet valve 149 are unitarily formed as a one-piece valve 150. In another example, the port valve 151 and the inlet valve 149 can be formed separately and subsequently joined or combined to form a single integrated valve 150. For example, the port valve 151 and the inlet valve 149 can be welded, glued, or joined using a snap fit arrangement in which one component is slid into another component. The valve 150 may be made using an elastomer, a TPE, an elastic biocompatible material, or combinations thereof. In one example, the valve 150 is made from a silicone material. In some examples, a metallic spring or other biasing material may be incorporated with the valve 150 to facilitate opening and/or closing of the port valve 151, the inlet valve 149, or both valve components 151, 149. When the valve 150 is formed from two or more different components, the different components can be made from different material properties, such as different material types and/or different durometers.

The valve 150 has a generally cylindrical body extending between an open distal end and a proximal end with a septum. Internally, a bore permits fluid communication between the open distal end and the split septum of the inlet valve 149. The split septum can be selectively opened to allow a flow path to flow between the distal and proximal ends. When positioned inside the interior cavity 127 of the catheter body 121, the port valve 151 seals off the interface between the injection port 124 and the interior cavity 127 of the catheter body 121 by presenting a surface, when in the normal expanded state, that covers the internal opening at the interface between the injection port 124 and the interior cavity 127 to prevent fluid from entering the interior cavity 127 from the injection port 124. The surface of the port valve 151 also prevents fluid from passing through the interior cavity 127 from either the distal end 123 or the proximal end 126 of the catheter body 121 from entering the lumen of the injection port 124. The port valve 151 is sized and shaped to be received within the interior cavity 127 of the catheter body 121, but can be sized slightly larger, such as larger in diameter, than the interior cavity 127 to provide a tight fit inside the interior cavity 127 and particularly at the opening between the injection port 124 and the interior cavity 127. The septum at the port valve 151 is normally closed to prevent fluid from passing through the interior cavity of the catheter hub via the proximal opening of the catheter body. When the septum is activated, such as when pushed by a male medical implement, fluid is permitted between the proximal and distal ends of the catheter hub.

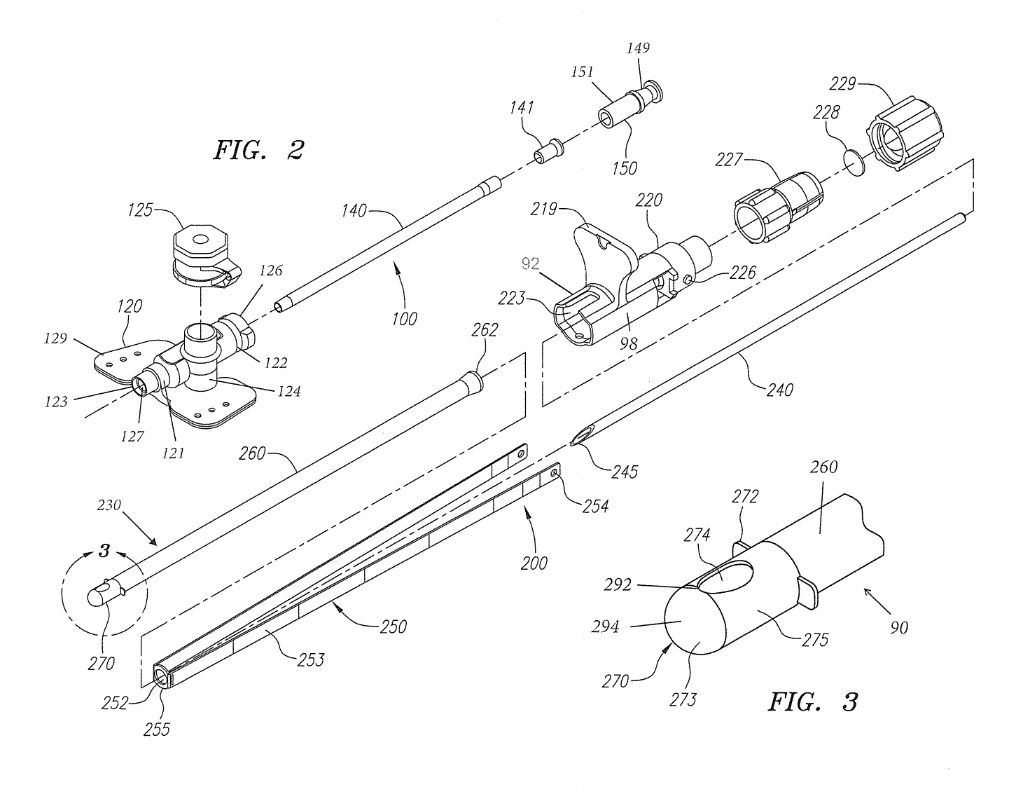

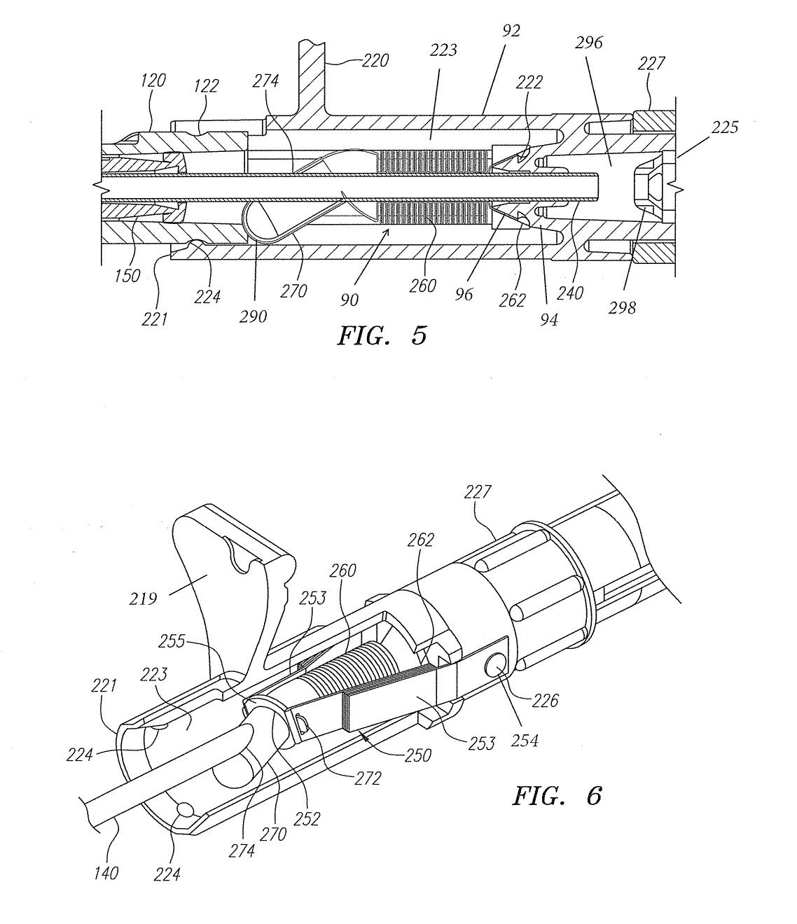

The needle hub unit 200 comprises a needle hub 220, and a needle 240 extending distally from the needle hub body 98. The needle hub 220 comprises a blood flashback chamber and a vent plug, which is typically attached to the proximal end of the catheter hub. As shown in FIGS. 3-6, a needle guard 90 can be included to prevent needle stick injuries following successful venipuncture. In an example, the needle guard 90 comprises an elastic sleeve 260. A tip cover 270 is attached to a distal end of the elastic sleeve 260 of the needle guard. The needle guard 90 can be positioned in a distal shroud 92 of the needle hub 220. The distal shroud 92 can be a continuous cylinder or can comprise a slot, as show in FIG. 2. A ring 262 (FIG. 5) can attach a proximal end of the sleeve 260 of the needle guard 90 to a distal nose section 94 located in the interior cavity 223 of the distal shroud 92 of the needle hub 220. In an example, the ring 262 can attach to a coupling 96 which is attached to a proximal end of the elastic sleeve 260. As shown, the coupling 96 can embody a conical shaped structure and the ring 262 can embody one of a male and female detents for engaging the other one of the male and female detents 222 on the distal nose section 94 of the needle hub 220. The coupling 96 and the ring 262 can be integrally formed.

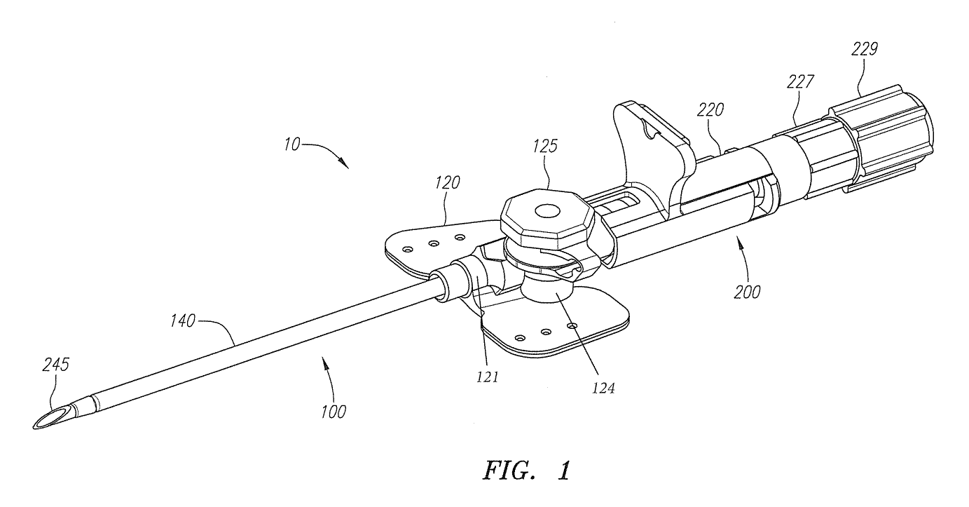

The needle 240 extends through the distal nose section 94 of the needle hub body 98 and through the sleeve 260 and out the tip cover 270 to pass through the septum and the bore of the valve 150, the catheter hub 120, and the catheter tube 140 in a ready to use position, as shown in FIG. 1 and partially in FIGS. 5 and 6. In the ready to use position, the catheter assembly 10 is ready for vascular access. The needle tip 245, such as the beveled end, of the needle 240 extends distally of the opening at the distal end of the catheter tube 140 in the ready to use position. The tip cover 270 is configured to secure the needle tip 245 from accidental needle sticks following use. For example, the tip cover 270 is configured to prevent the needle tip 245 from puncturing through a membrane to expose the needle tip 245 after the needle tip is covered by the tip cover 270, as further discussed below. In one embodiment, the flashback chamber of the needle hub 220 can be separately formed and subsequently attached to a distal needle hub portion. With reference to FIGS. 3 and 4, an air vent housing 227can attach to a proximal end of the needle hub 220 and a closing cap 229 securing a filter 228 between the closing cap 229 and the air vent housing 227. In one example, the needle hub body 98 includes a proximal cylinder sized and shaped to project into a bore of the air vent housing 227. In some examples, the connection can reverse with the air vent housing having a cylinder sized and shaped to project into a bore of the needle hub body 98. The connection between the needle hub body and the air vent housing can have an interference fit and can optionally be glued or welded together. In other examples, threaded engagement may be used to secure the two together. Raised projections or other surface features may be included with the air vent housing 227 to facilitate gripping. The closing cap 229 can couple to the air vent housing 227 in a similar manner.

With reference again to the needle guard 90, the elastic sleeve 260 can encase or cover the needle 240 when the needle 240 is withdrawn from the ported catheter unit 100. The sleeve 260 can also capture blood to prevent spilling or droplets from falling on the ground or other surfaces. In one embodiment, the sleeve 260 is a long hollow tubular structure that is collapsible or compressible into random or defined folds to store elastic energy in the ready to use position. In one example, the sleeve 260 is made of an elastic material, such as a compressible syntactic rubber capable of storing elastic energy when compressed in the ready to use position. In another example, the sleeve 260 is made from a neoprene or isoprene material via transfer molding or injection molding. Other elastomer and thermoplastic elastomer materials are contemplated for making the sleeve 260.

With reference to FIGS. 3, 5, and 6, the proximal end of the sleeve 260 of the needle guard 90 is attached to the distal nose section 94 in the interior cavity 223 of the distal shroud 92 using a ring 262. The ring 262 can snap into mating surfaces on the distal nose section 94. In an example, the ring 262 has a conical shape for coupling to a conical shape distal nose section 94. In other examples, the ring and the distal nose section can embody other shapes, such as being generally cylindrical. The proximal end of the sleeve 260 can attach to the ring 262 by mechanical means, such as clamping, fastening, or adhesive. In some example, the ring 262 is unitarily formed with the sleeve 260 and is made from the same material as the sleeve. The distal end of the sleeve 260 can attach to the tip cover 270, which is further discussed below, by mechanical means, such as by clamping, fastening, or adhesive. In the ready to use position, the needle 240 projects through a side opening 274 of the cylinder of the tip cover 270 and causes the tip cover 270 to cant off-axis from the lengthwise axis of the needle guard 90. As more clearly shown in FIGS. 4 and 5, the tip cover 270 is slanted and contacts the interior surface of the distal shroud 92 as well as the proximal end of the catheter hub. Under the elastic energy of the sleeve 260, the tip cover 270 is urged to return to its normal linear in-line position but for the presence of the needle 240. Thus, the tip cover 270, when canted, exerts a force onto or against the needle 240 and vice versa. Accordingly, an aspect of the present disclosure is understood to include a needle guard comprising a compressible sleeve attached to a cylinder and wherein the needle guard has a lengthwise axis and wherein a side opening is provided on the cylinder at a location away from the lengthwise axis. In an example, the side opening can be tangent to the lengthwise axis of the needle guard. The cylinder can further comprise a distal end and wherein the distal end, at a location along the lengthwise axis of the needle guard, is solid. In another example, the distal end, at a location along the lengthwise axis of the needle guard, can comprise an opening but wherein a perimeter defining the opening is smaller in diameter than the diameter of the needle shaft. This arrangement allows the needle to only pass through the side opening 274.

From the position shown in FIGS. 4-6 and following successful venipuncture, the needle 240 can be retracted away from the catheter hub 120. During the retraction procedure, the tip cover 270 remains in contact with the proximal end of the catheter hub but the compressible sleeve 260 expands lengthwise as the needle retracts. Once the needle tip 245 moves proximally of the side opening 274 on the needle guard 90, the stored elastic energy of the sleeve 260 is released and the tip cover 270 returns to its original configuration, in-line with the lengthwise axis of the needle guard, similar to that shown in FIG. 3. The needle 240 and the needle tip 245 are now positioned inside the sleeve 260 and the tip cover 270 and the needle device can be said to be in a protected position, shielded position, or secured position.

Because the elastic sleeve 260 is compressed in the ready to use position and expands in the protected position to lengthen from a first length to a second length, which is longer than the first length, the sleeve 260 can act as a compression spring, which is compressed in the ready to use or first position and springs back to its original shape during needle 240 withdrawal towards the secured or second position. The elastic properties of the elastic sleeve 260 will allow the sleeve 260 to return to its original shape when the needle 240 is withdrawn and the needle tip moves proximally of the distal edge of the side opening and the tip cover 270 tilts back to an axis substantially in line with the lengthwise axis of the needle guard to secure the needle tip 245 inside the tip cover 270. Thus, when the tip cover 270 is aligned with the needle 240, the nose section 290 (FIG. 5) of the tip cover 270 will un-bias away from the interior surface of the distal shroud 92 to cover the needle tip 245.

The sleeve 260 is understood to be movable or expandable during the retraction step of the needle away from the catheter tube and the catheter hub, such as following successful venipuncture. In an example, the needle hub can completely separate from the catheter hub as the needle retracts in the proximal direction and wherein the needle guard 90 continues to contact both the needle hub and the catheter hub.

In the illustrated embodiment, a folded mechanism 250 is incorporated to act as a tether for the tip cover 270, such as to prevent the sleeve 260 from being stretched too far and possibly tearing the sleeve or causing the sleeve to separate from the distal nose section 94 of the needle hub 220. The folded mechanism 250 has a plurality of folded legs 253, which are interconnected. The folded mechanism 250 can have a first end anchored to the needle hub 220 and a second end capturing, fixing, securing or engaging the tip cover 270, thereby forming a restraint that limits the separation between the tip cover and the needle hub. In an example, a single folding mechanism 250 is incorporated as a tether. In another example, two folding mechanisms on two different sides of the sleeve 260 are used to provide the restraint. In an example, a nose plate 255 is used to attach to an end of each folded mechanism 250 with the other end of the folded mechanism attached to the needle hub. The nose plate 255 (FIG. 6) has a perimeter defining a hole 252 therein for receiving the tip cover 270. The folded mechanism 250 has one end extending from the nose plate 255 and another end attach to an anchoring point on the needle hub 120, such as a nipple 226 extending from the catheter hub 120. In the illustrated embodiment, folded mechanisms 250 extend proximally from the nose plate 255 and each comprising a hole 254 at the proximal free end attaching to a respective nipple 226 at opposite sides of the needle hub 220. The legs 253 of the two folded mechanisms should be unyielding but able to fold over each other to occupy a small volume, such as inside the distal interior cavity 223 of the needle hub 200. In some examples, the folded mechanisms 250 are single or multi-strand strings rather than folded legs.

With reference again to needle guard 90 of FIG. 3, the tip cover 270 includes a hollow elongated body 275 and a nose section 273 formed at a distal end of the elongated body 275. In an example, the elongated body 275 can be a cylinder and the nose section can be dome-shaped. One or more flanges 272 can extend laterally from or near a proximal end of the elongated body 275, and a side opening 274 located off-axis from the lengthwise axis of the needle guard 90. In one example, the side opening 274 is generally round. In another example, the side opening is oval or elliptical in shape. In yet other examples, the side opening is square, rectangle, or triangular in shape. Other shaped openings are contemplated, including non-symmetrical, random, polygonal, etc. The perimeter of the side opening can be smooth or radiused. A distal section or edge 292 of the perimeter of the side opening 274 can be recessed or positioned proximally of the distal tip 294 of the nose section 273.

When installed over a needle, the needle cannot project through the side opening when the tip cover 270 is in the normal in-line configuration of FIG. 3. The needle can pass through the side opening 274 when the tip cover 270 is canted or off-axis from the lengthwise axis of the needle guard 90, as shown in FIGS. 4-6. Said differently, the tip cover 270 has a lengthwise axis that is common or in-line with the lengthwise axis of the needle guard 90. When the axis of the tip cover is in-line, the needle cannot project through the side opening. When the axis of the tip cover is off-axis or not common with the lengthwise axis of the needle guard 90, the needle can project through the side opening 274. In one example, the side opening is formed only in the body or hollow cylinder 275 of the tip cover 270. Thus, when the needle tip 245 is moved inside the tip cover 270, the needle tip 245 is unable to exit the tip cover 270 through the side opening 274, unless the tip cover 270 is manually tilted off-axis again.

In an example, the width of the side opening 274 should be at least slightly larger than the diameter of the needle 240. The length of the side opening 274 depends on the pitch or degree of tilt of the tip cover 270 when positioned inside the interior cavity 223 of the distal shroud 92 of the needle hub 220. The tip cover 270 is configured to pass through the hole 252 of the nose plate 255 until the flanges 272 of the tip cover 270 abut against the nose plate 255 to prevent the tip cover 270 from passing completely through the hole 252 of the nose plate 255. In another example, the elongated body 275 of the tip cover 270 tapers outwardly so that the diameter of the tapered portion restricts the amount of insertion of the tip cover 270 into the hole or opening 252 of the nose plate 255. The tip cover 270 should be made of a material that is able to resist needle punctures. In one example, the tip cover 270 is made of metal material, such as stainless steel formed using a metal stamping process. In another example, the tip cover 270 is made of a rigid plastic such as POM or ABS. In still other examples, the tip cover 270 is made from an elastomeric material and has a metal insert, metal sleeve, or metal flange located at the distal tip 294 of the nose section 273. For example, the metal insert, sleeve, or flange can be adhered to the inside surface of the nose section 273 or embedded into the nose section 273 by insert molding, as examples. The insert, sleeve, or flange can also be made from a hard plastic instead of a metal.

With reference again to FIGS. 4-6, a sectional view of the catheter assembly 10 is shown with the needle hub 220 being generally cylindrical with a grip flange 219 extending laterally and outwardly from an outer surface of the needle hub 220. A latching means, such as one or more bumps 224, can be provided at a distal opening 221 of the distal shroud 92 of the needle hub 220 to engage the proximal end of the catheter hub 120 and latch the needle hub 220 to the catheter hub 120. For example, the spaced apart bumps 224 can be incorporated to grip against the external threads, such as a shoulder or ledge of the threads, of the catheter hub 120 to retain the needle hub to the catheter hub. As previously mentioned, the bumps 224 can also engage corresponding recesses on the catheter hub. The grip flange 219 is configured to aid the user in inserting the needle 240 into a vasculature of a patient, and withdrawing the needle hub 220 from the catheter hub after successful venipuncture.

The needle hub 220 has a first interior cavity 296 and a second interior cavity 225 located within the male projection 298 of the air vent housing 227. The first interior cavity 296 and the second interior cavity 225 are in fluid communication with one another via an orifice or opening at an end of the male projection 298. A proximal end of the needle 240 is in fluid communication with the first interior cavity 296 and the second interior cavity 225. The needle 240 extends through the distal nose section 94, through the needle guard 90, and through the catheter unit 100 with the needle tip 245 extending out a distal end of the catheter tube 140.

Referring again to FIG. 5, a proximal end of the sleeve 260 is fixed to the ring 262 and the ring 262 can engage the undercut 222, as previously discussed. The distal end of the sleeve 260 is secured to the tip cover 270. For example, the coupling 96 and the tip cover 270 can be fixed to the sleeve 260 by adhesive, bonding and/or mechanical engagement. In an example, the sleeve 260 can be a compressible or collapsible hollow tube. The sleeve 260 can be made of an elastic material, such as a compressible syntactic rubber capable of storing elastic energy when compressed in the ready to use position. In some examples, exterior of the sleeve 260 is provided with pre-formed kinks or pleats so that the elastic sleeve 260 compressed along the kinks or pleats when in the ready to use position shown. In other examples, no kinks or pleats are provided and the elastic sleeve 260 simply compress in random folds.

In the ready to use position, the needle hub 220 is removably attached to the catheter hub 120 by engaging the one or more bumps 224 of the needle hub 220 with the one or more recesses 122 defined in an outer surface of a proximal portion of the catheter hub 120. The engagement between the needle hub 220 and the catheter hub 120 temporarily secures the needle hub 220 to the catheter hub 120. In other examples, the bumps 224 simply rest against a shoulder or ledge on the external threads of the catheter hub to retain the needle hub to the catheter hub. The needle 240 passes through the compressible sleeve 260 and the tip cover 270 through the side opening 274 to the catheter unit 100, where it then passes through the septum of the inlet valve 150 and out the port valve 151 located inside the catheter body 121 and out the catheter tube 140 with the needle tip 245 exposed.

In the ready to use position, the tip cover 270 is tilted off-axis front the lengthwise axis of the needle guard 90 and abuts against the proximal end of the catheter hub 120 to maintain the sleeve in the compressed or collapsed state storing the elastic energy. The tip cover 270 also contacts the interior surface of the distal shroud 92 in the ready to use position. The air vent housing 227, filter 228, and closing cap 229 cooperatively allow gas to vent from the needle 240 after successful venipuncture has occurred. Once the needle 240 has successfully punctured a blood vessel of a patient, the blood enters the first interior cavity 296 and if necessary the second interior cavity 225 via the opening of the male projection 298. The filter 228 can be a hydrophobic filter that allows air to vent but traps blood within the second interior cavity 225.

Referring again to FIG. 6, the tip cover 270 is tethered to the needle hub 220 via the folded mechanism 250. In one embodiment, the hole 254 at the first end of each of the folded mechanism is placed over a nipple 226 on the needle hub 220 and optionally heat welded thereto to fix the first end of the folded mechanism 250 to the needle hub 220. In other examples, the needle hub is provided with a hole and the folded mechanism is provide with a nipple or projection for engaging the hole. The second end or distal end of each folded mechanism 250 is attached to the nose plate 255 and/or a corresponding flange 272 on the tip cover 270.

In another embodiment, the folded mechanism 250 is integrally formed with the nose plate 255. The nose plate 255 has a perimeter defining a hole 252 in a center thereof to receive the tip cover 270, as previously discussed. The size or diameter of the hole 252 is slightly larger than the body portion 275 of the tip cover 270, but less than a distance between opposite ends of the two flanges 272. In another example, a single continuous flange is provided with the tip cover instead of spaced apart flanges. The nose 273 of the tip cover 270 extends through the hole 252 of the nose plate 255 until distal surfaces of the flanges 272 abut against the proximal surface of the nose plate 255. The size of the hole 252 thus prevents the tip cover 270 from passing completely through the hole 252.

In yet another embodiment, slots can be formed with the nose plate 255 or the folded mechanism 250, which acts as a tether, to accommodate the flanges 272 passing therethrough to secure the tip cover 270 to the nose plate 255.

In still yet another embodiment, the distal end of the folded mechanism 250 can attach directly to the tip cover 270 at the flange 272 or to the surface of the tip cover. The tether can also be integrally formed with the tip cover 270, thereby eliminating the need for the nose plate 255 and/or the flanges 272.

After successful venipuncture and upon retraction of the needle hub 220 from the catheter hub 120, the bumps 224 on the needle hub 220 disengage from the recess 122 of the catheter hub 120 and the sleeve 260 begins to expand as the needle hub 220 is withdrawn away from the catheter hub 120. The tip cover 270 remains in abutting contact with the proximal end of the catheter hub. The tip cover 270 slides relative to the needle 240 as the elastic sleeve 260 releases its stored energy and the tip cover 270 moves relative to the needle 240 until the sleeve 260 is fully expanded and the needle tip 245 moves proximally of the distal edge or distal section 292 (FIG. 3) of the side opening 274 and eventually inside the tip cover 270 to be shielded by the tip cover 270, also called the secured position or shielded position.

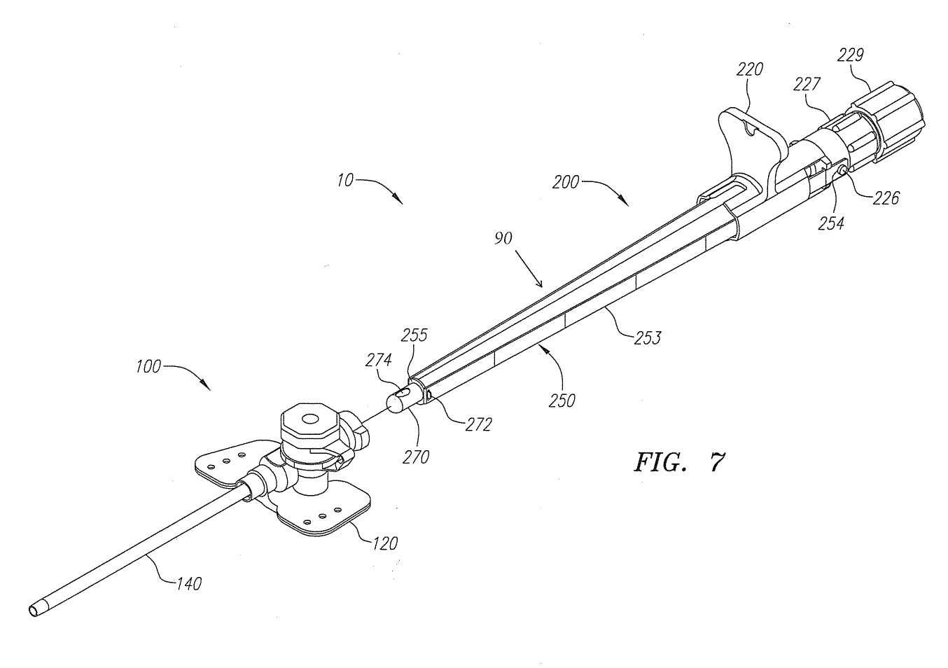

FIG. 7 shows the needle guard 90 in the shielded position and the needle tip located inside the tip cover 270. The two folded mechanisms 250 are shown expanded to restrict further expansion by the sleeve 260. Optionally only one folded mechanism 250 is used. Because the sleeve 260 is elastic, the folded mechanisms 250 prohibit a scenario where the tip cover 270 is moved distally by stretching the sleeve 260. If stretching is allowed, the needle tip 245 can come in contact with the sleeve 260 and can puncture through the sleeve. The hard material of the tip cover 270, or an insert made from a hard material placed at the tip cover as previously discussed, can prevent the needle tip 245 from puncturing through the tip cover 270.

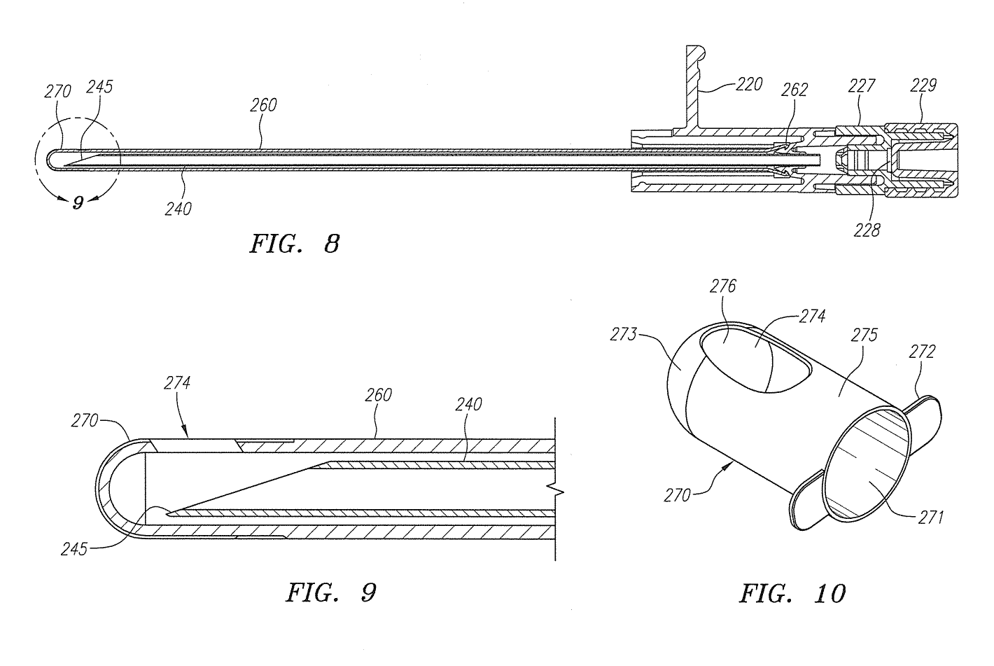

Referring now to FIGS. 8 and 9, as the tip cover 270 moves distally under the elastic force of the sleeve 260 until the needle tip 245 slips through the side opening 274, the tip cover 270 rotates back into an in-line position in which the axis of the tip over coaxially aligns with the lengthwise axis of the needle guard 90. In this position, the needle 240 and needle tip 245 cannot easily exit through the side opening 274 without external manipulation. Therefore, the needle tip 245 is safely shielded inside the tip cover 270, which does not have an opening in-line with the lengthwise axis of the needle guard for the needle tip to re-emerge. In another example, an opening is provided at a distal end of the tip cover and in-line with the lengthwise axis of the needle guard. However, if the opening is provided, the will have a smaller diameter than the diameter of the needle shaft to prevent the entire tip and large sections of the needle shaft from re-emerging out the tip cover.

The embodiment of the tip cover 270 illustrated in FIG. 10 has a hollow elongated body 275 with one or more flanges 272 and an opening 271 at a proximal end thereof, and a nose end 273 at a distal end of the elongated body 275. The side opening 274 is configured to allow the needle to extend through both the proximal opening 271 and the side opening 274 when the tip cover 270 is in the ready to use position. That is, because the side opening 274 is at a side of the tip cover 270 away from the lengthwise axis of the tip cover 270, the tip cover 270 should tilt and the side opening 274 at least partially aligned with the lengthwise axis of the tip cover for the needle to project therethrough. Further, because the tip cover 270 is connected to the sleeve 260, when the tip cover is tilted, the sleeve 260 can be loaded with stored energy and will tend to straighten the tip cover 270 to a position of least resistance. There should be minimal friction between the needle 240 and the side opening 274 and proximal opening 271 of the tip cover 270 to ensure the tip cover 270 can slide along the needle 240 when moving to the secured position. In one example, a coating can be applied to reduce friction between the needle 240 and the tip cover 270. In another example, the tip cover 270 can be made of a material that has a relatively low and suitable coefficient of friction. In yet another example, the contact surfaces with the needle 240, such as around the side opening 274, and the opening 271 are rounded, radiused, or smooth to minimize friction.

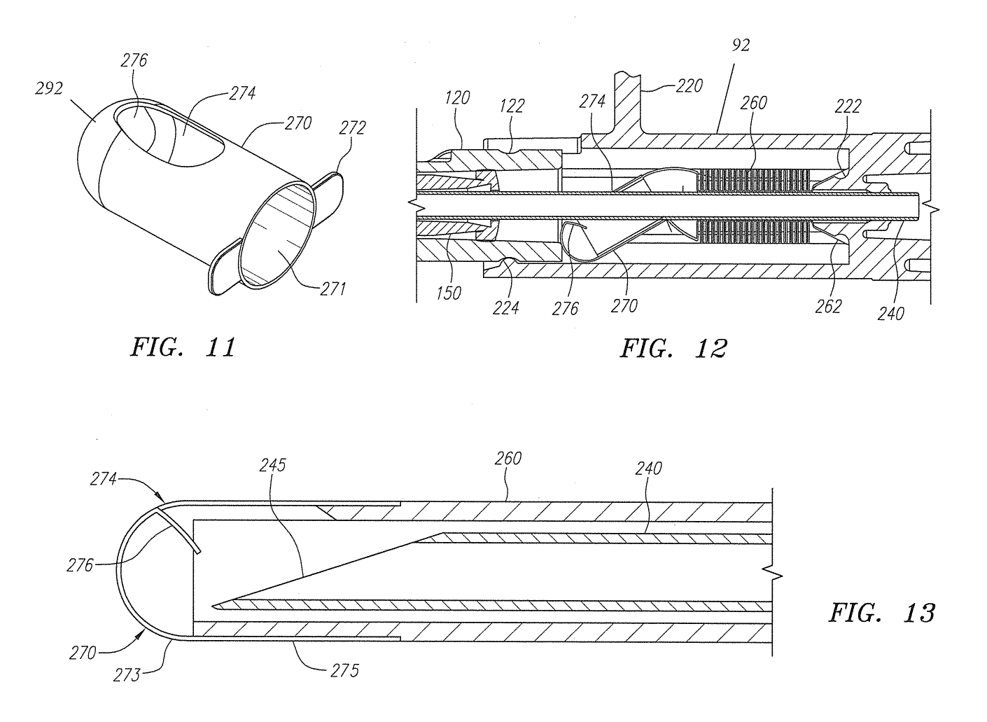

FIG. 11 illustrates another embodiment of the tip cover 270 which is similar to the tip cover of FIG. 10, except that a flap 276 is provided along the distal section 292 of the side opening 274 and extends proximally into the interior cavity of the tip cover 270. The flap 276 acts as a restriction and prevents or thwarts the needle tip 245 from extending back out the side opening 274 once the needle hub unit 200 is in the secured or shielded position.

Referring to FIG. 12, the tip cover 270 of FIG. 11 is shown positioned inside the distal shroud 92 of the needle hub 220 and is tilted to allow the needle 240 to pass through the side opening in the ready to use position. The flap 276 is shown and is configured so as not to interfere with the sliding of the tip cover 270 relative to the needle 240. The flap 276 can be flexible or semi-rigid and configure to restrict the needle tip's ability to re-emerge out the side opening 274 after the shielded position.

In the secured position shown in FIG. 13, the needle tip 245 is secured inside the tip cover 270. The flap 276 helps prevent the needle from exiting the side opening 274. Therefore, the needle tip 245 is safely secured in the tip cover 270.

Methods of making and of using the IV catheter assembly and their components described elsewhere herein are contemplated and are considered within the scope of the present disclosure.

The above description presents various embodiments of the present invention, and the manner and process of making and using them, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention.

What is claimed is:

1. A catheter assembly, comprising:
a catheter tube extending distally from a distal end of a catheter hub;
a needle hub having an interior cavity;
a needle guard having a lengthwise axis and comprising a sleeve having a proximal end attached to the needle hub and a tip cover attached at a distal end of the sleeve; the sleeve surrounding at least a portion of the needle and the tip cover comprising a nose section and a perimeter defining a side opening located off-axis from the lengthwise axis of the needle guard;
a needle with a needle tip projecting through the sleeve, the side opening of the tip cover, the catheter hub, and the catheter tube in a ready to use position with the needle tip extending distally of a distal opening of the catheter tube.

2. The catheter assembly of claim 1, further comprising a valve positioned in an interior cavity of the catheter hub.

3. The catheter assembly of claim 2, wherein the catheter hub is a ported catheter with an injection port.

4. The catheter assembly of claim 3, further comprising a port cap assembly capping the injection port of the ported catheter.

5. The catheter assembly of claim 3, wherein the valve has a port valve sealing the injection port from an interior cavity of the catheter hub, and an inlet valve sealing fluid flow from a proximal opening of the catheter hub.

6. The catheter assembly of claim 1, wherein the sleeve is elastic and is compressed in the ready to use position.

7. The catheter assembly of claim 6, wherein the sleeve is compressed and bent near a distal end of the sleeve.

8. The catheter assembly of claim 6, wherein the sleeve is made of a compressible syntactic rubber, neoprene, or isoprene.

9. The catheter assembly of claim 6, wherein the sleeve is attached to the needle hub via a ring fixed to the proximal end of the sleeve and inside a distal shroud of the needle hub.

10. The catheter assembly of claim 2, wherein the tip cover slides relative to the needle from the ready to use position to a secured position where the needle tip enters into the tip cover through the side opening.

11. The catheter assembly of claim 10, wherein the side opening has a width larger than a diameter of the needle.

12. The catheter assembly of claim 10, wherein a perimeter of the side opening is smooth to minimize friction between the needle and the tip cover.

13. The catheter assembly of claim 10, wherein the tip cover abuts against the catheter hub in the ready to use position.

14. The catheter assembly of claim 13, wherein the tip cover abuts against the catheter hub and the sleeve is attached to the needle hub in the ready position and during retraction of the needle away from the catheter hub.

15. The catheter assembly of claim 10, wherein the tip cover further comprises a flap extending in a proximal direction inside the tip cover from a distal end of the side opening, said flap being configured for preventing the needle tip from emerging out of the side opening from the secured position.

16. The catheter assembly of claim 10, wherein the tip cover is made of metal, POM or ABS.

17. The catheter assembly of claim 10, wherein the tip cover is tethered to the needle hub.

18. The catheter assembly of claim 17, wherein the tether is a folded mechanism attached to the tip cover and the needle hub.

19. The catheter assembly of claim 18, wherein the folded mechanism is attached to a nose plate, which is attached to the tip cover.

20. The catheter assembly of claim 19, wherein the folded mechanism comprises a plurality of legs folded in the interior cavity of the needle hub in the ready to use position.

21. The catheter assembly of claim 18, wherein the tip cover and the folded mechanism are integrally formed.

22. The catheter assembly of claim 14, further comprising an air vent housing attached to a proximal end of the needle hub, a closing cap attached to a proximal end of the air vent housing, and a filter sandwiched between the air vent housing and the closing cap.

23. A method of making a catheter assembly, comprising:
providing a catheter unit comprising a catheter hub with a hub body, a catheter tube extending distally from a distal end of the catheter hub, and a valve positioned in an interior cavity of the catheter hub;
providing a needle hub unit comprising a needle hub having an interior cavity, a needle with a needle tip extending distally from the needle hub; and a needle guard having a lengthwise axis coupled to the needle hub; said needle guard comprising an elastic sleeve having a proximal end coupled to the needle hub, and a tip cover attached to a distal end of the sleeve, said tip cover having a nose section and a side opening located off-axis from the lengthwise axis of the needle guard;
extending the needle through the elastic sleeve and out the side opening on the tip cover, through the valve, and through the catheter tube with the needle tip extending distally of a distal opening of the catheter tube in a ready to use position;
compressing the elastic sleeve against the catheter hub and the needle hub in the ready to use position; and
tilting the nose section of the tip cover from the lengthwise axis of the needle guard.

24. The method of claim 23, further comprising a side injection port extending from the hub body.

25. The method of claim 24, further comprises a port cap assembly capping an injection port.

26. The method of claim 23, further comprising sliding the tip cover relative to the needle from the ready to use position to the secured position.

27. The method of claim 23, wherein the sleeve is attached to the needle hub via a ring at the proximal end of the sleeve and the distal nose section in the interior cavity of the needle hub.

28. The method of claim 23, wherein the tip cover is tethered to the needle hub.

29. The method of claim 28, wherein the tip cover is tethered with a folded mechanism that is attached to a flange extending from the tip cover and to a heat weld nipple extending from the needle hub.

30. The method of claim 29, wherein the folded mechanism is attached to a nose plate which is coupled to the tip cover.

31. The method of claim 30, wherein the nose plate has a perimeter defining an opening having the tip cover projecting therethrough.

32. The method of claim 28, wherein the tip cover and the folded mechanism are integrally formed.

33. The method of claim 23, wherein the tip cover further comprises a flap extending proximally inside the tip cover from a distal end of the side opening, said flap being configured for preventing the needle tip from coming out of the side opening.

34. The method of claim 23, wherein the side opening has a width larger than a diameter of the needle.

35. The method of claim 23, wherein a perimeter of the side opening and a perimeter of the opening is smooth to minimize friction between the needle and the tip cover.

36. The method of claim 23, wherein the tip cover abuts against the catheter hub in the ready to use position.

37. The method of claim 28, wherein the tip cover further comprises a flap extending proximally inside the tip cover from a distal end of the side opening, said flap being configured for preventing the needle tip from coming out of the side opening.

38. The method of claim 23, further providing an air vent housing attached to a proximal end of the needle hub, a closing cap attached to a proximal end of the air vent housing, and a filter sandwiched between the air vent housing and the closing cap.
